(19) United States
(12) Patent Application Publication
(10) Pub. No.: US 2024/0197995 A1
(43) Pub. Date: Jun. 20, 2024

(54) A COMPONENT AND A SUB-ASSEMBLY FOR A MEDICAMENT DELIVERY DEVICE

(71) Applicant: SHL Medical AG, Zug (CH)

(72) Inventor: Daniel CARLSSON, Nacka Strand (SE)

(21) Appl. No.: 18/553,605

(22) PCT Filed: Apr. 19, 2022
(86) PCT No.: PCT/EP2022/060235

§ 371 Date: Oct. 2, 2023

(30) Foreign Application Priority Data
Apr. 28, 2021 (EP) 21171044.7

(51) Int. Cl. A61M 5/20; A61M 5/315

(52) U.S. Cl. CPC A61M 5/2033; A61M 5/31501; A61M 2005/2013; A61M 2205/6045

(57) ABSTRACT

A plunger rod (2) for a medicament delivery device, the plunger rod (2) comprising: a plunger rod body (21) extending along a second longitudinal axis (L2) between a proximal end and a distal end; a key (22) attached to the proximal end of the plunger rod body (21); wherein the key (22) is configured to fit into a key slot (13a) on a cassette housing (1), such that when the key (22) and the key slot (13a) are matched, the key (22) followed by the plunger rod body (21) can enter into the cassette housing (1) through the key slot (13a).

### TECHNICAL FIELD

The present disclosure generally relates to components and sub-assemblies for medicament delivery devices such as reusable autoinjectors or injectors with two detachable parts, and particularly to components and/or sub-assemblies comprising a key feature or a key slot feature.

### BACKGROUND

Medicament delivery devices such as pen type manual injectors or auto-injectors are generally known for the self-administration of a medicament by patients without formal medical training. For example, patients suffering from diabetes may require repeated injections of insulin, or patients may require regular injections of other types of medicaments, such as a growth hormone.

Some medicament delivery devices have two parts which are interconnected during use. One part often receives a medicament container which contains a medicament, such as a cartridge or a syringe; this part usually can be called a cassette unit. The other part often comprises a driver for actuating expelling the medicament.

Medicament delivery devices designed in this way are commonly reusable. The cassette unit is a disposable part, and the other part with the driver usually is reusable. The driver may comprise a power unit such as a spring or a motor and thus can be re-used for many medicament deliveries. The driver may be designed according to the features of the specific medicament, e.g. volume, viscosity, medicament delivery time. Therefore, if a user accidentally attaches a cassette unit to a “wrong” driver part, the treatment may be less efficient. Furthermore, the reusable driver part may comprise an electric power source, if the user attaches the cassette unit to a driver part when a battery of the power source is low, e.g. not enough to perform a complete medicament delivery operation.

It has been appreciated that solutions for preventing the user from mixing up the cassette unit and the driving part could be advantageous.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

A first aspect of the invention is hence provided with a cassette housing for a medicament delivery device cassette unit, the cassette housing comprising: a main body extending along a first longitudinal axis between a proximal end and a distal end. The main body defines an interior chamber for receiving a medicament container; a distal cover attached to the distal end of the main body. The distal cover comprises a covering wall extending across the distal end of the interior chamber of the main body in a direction transverse to the first longitudinal axis. The covering wall comprises a key slot configured to receive a key of a plunger rod of the medicament delivery device, such that when the key and the key slot are matched, the key of the plunger rod followed by a plunger rod body of the plunger rod can enter into the cassette housing through the key slot.

A second aspect of the invention is provided with a plunger rod for a medicament delivery device, the plunger rod comprising: a plunger rod body extending along a second longitudinal axis between a proximal end and a distal end; a key attached to the proximal end of the plunger rod body. The key is configured to fit into a key slot on a cassette housing, such that when the key and the key slot are matched, the key followed by the plunger rod body can enter into the cassette housing through the key slot.

A third aspect of the invention is provided with a sub-assembly comprising the cassette housing as defined above and the plunger rod as defined above.

The cassette housing and the plunger rod therefore can prevent a medicament delivery operation in which the user attaches the cassette housing to a “wrong” distal body of the medicament delivery device.

Preferably, according to another embodiment, the sub-assembly comprises a distal body extending along a third longitudinal axis between a proximal end and a distal end. The distal body is configured be attached to the cassette housing. The plunger rod is arranged in the distal body (3) and is telescopic relative to the proximal end of the distal body.

Preferably, according to another embodiment, the cassette housing comprises a proximal engaging member attached at the distal end of the main body of the cassette housing.

Preferably, according to another embodiment, the distal body comprises a distal engaging member attached to the proximal end of the distal body.

Preferably, according to another embodiment, the cassette housing is attached to the distal body when the proximal engaging member is fastened to the distal engaging member

Preferably, according to another embodiment, the proximal engaging member is configured to be fastened to the distal engaging member in the direction of the third longitudinal axis through a bayonet connection.

Preferably, according to another embodiment, the proximal engaging member is a bayonet slot; and the distal engaging member is a bayonet protrusion.

Alternatively, according to another embodiment, the proximal engaging member is a bayonet protrusion; and the distal engaging member is a bayonet slot.

Alternatively, according to another embodiment, the proximal engaging member is configured to be fastened to the distal engaging member in the direction of the third longitudinal axis through a thread connection.

Preferably, according to another embodiment, the proximal engaging member is a thread; and the distal engaging member is a counter thread.

Alternatively, according to another embodiment, the proximal engaging member is a thread; and the distal engaging member is a protrusion.

Alternatively, according to another embodiment, the proximal engaging member is a protrusion; and the distal engaging member is a thread.

Alternatively, according to another embodiment, the proximal engaging member is configured to be fastened to the distal engaging member in the direction of the third longitudinal axis through a snap-fit connection.

Preferably, according to another embodiment, the proximal engaging member is a distally extending flexible arm from the distal end of the cassette housing; the flexible arm comprises a transverse head transversal to the longitudinal axis, and the distal engaging member is an aperture opening towards the direction transverse to the longitudinal axis, the aperture is arranged on the proximal end of the distal body.

Alternatively, according to another embodiment, the proximal engaging member is an aperture opening towards the direction transverse to the longitudinal axis, the aperture is arranged on the distal end of the cassette housing, and the distal engaging member is a proximally extending flexible arm from the proximal end of the distal body; the flexible arm comprises a transverse head transversal to the longitudinal axis.

Alternatively, according to another embodiment, the proximal engaging member is fastened to the distal engaging member through a press-fit connection.

Alternatively, according to another embodiment, the distal engaging member is longitudinally positioned relative to the distal body more distal than the proximal end of the plunger rod relative to the distal body.

Preferably, according to another embodiment, the proximal engaging member is positioned closer to the proximal end of the cassette housing than a distal end of the distal cover; and the key of the plunger rod protrudes beyond at least a portion of the distal engaging member in the proximal direction of the distal body.

Alternatively, according to another embodiment, the proximal engaging member is positioned relative to the cassette housing at the same longitudinal position as the distal cover relative to the cassette housing.

Preferably, according to another embodiment, the key of the plunger rod protrudes beyond a longitudinal portion of the bayonet slot in the distal direction.

Alternatively, according to another embodiment, the key of the plunger rod protrudes beyond a longitudinal portion of the thread the distal direction.

Alternatively, according to another embodiment, the key of the plunger rod protrudes beyond a longitudinal portion of the aperture the distal direction.

Preferably, according to another embodiment, the plunger rod is movable from a first position where the key of the plunger rod is misaligned with the key slot to a second position where the key of the plunger rod is aligned with the key slot.

Preferably, according to another embodiment, the plunger rod is rotatable and/or linear movable along the longitudinal axis from a first position where the key of the plunger rod is misaligned with the key slot to a second position where the key of the plunger rod is aligned with the key slot.

Preferably, according to another embodiment, the plunger rod is configured to be temperately locked in the first position, so that the when the plunger rod in the first position, the plunger rod can be an indication of the status of the drive unit to the user.

Preferably, according to another embodiment, the key of the plunger rod is rotationally fixed to the plunger rod body; and the plunger rod body is rotatable around the second longitudinal axis from a first position to a second position.

Preferably, according to another embodiment, the key of the plunger rod is misaligned with the key slot of the distal cover of the cassette housing in the circumferential direction relative to the first longitudinal axis when the plunger rod is in the first position and at least a portion of the proximal engaging member is aligned with the distal engaging member in the direction of the first longitudinal axis.

Preferably, according to another embodiment, the key of the plunger rod of the distal body is misaligned with the key slot of the distal cover of the cassette housing when the plunger rod is in the first position and the bayonet protrusion is aligned with the longitudinal portion of bayonet slot.

Alternatively, according to another embodiment, the key of the plunger rod of the distal body is misaligned with the key slot of the distal cover of the cassette housing when the plunger rod is in the first position and the thread is aligned with the protrusion.

Alternatively, according to another embodiment, the key of the plunger rod of the distal body is misaligned with the key slot of the distal cover of the cassette housing when the plunger rod is in the first position and the flexible arm is aligned with the aperture.

Preferably, according to another embodiment, the distal body is configured to receive an energy source of the medicament delivery device, the energy source is configured to connect to the plunger rod for driving the plunger rod relative to the distal body.

Preferably, according to another embodiment, the energy source can be one of a motor, a torsion spring, a compression spring, a linear-movably band spring, and a gas canister.

Preferably, according to another embodiment, the plunger rod is configured to move along the third longitudinal axis in the distal direction relative to the distal body from a first position to a second position when the energy source of the medicament delivery device received in the distal body has sufficient power to carry out a medicament delivery operation.

Preferably, according to another embodiment, when the plunger rod is in the second distal position, the key of the plunger rod is positioned closer to the distal end of the distal body than the longitudinal portion of the bayonet slot.

Alternatively, according to another embodiment, when the plunger rod is in the second distal position, the key of the plunger rod is positioned closer to the distal end of the distal body than the longitudinal portion of the thread.

Alternatively, according to another embodiment, when the plunger rod is in the second distal position, the key of the plunger rod is positioned closer to the distal end of the distal body than the longitudinal portion of the aperture.

Alternatively, according to another embodiment, when the plunger rod is in the second distal position, the key of the plunger rod is positioned closer to the distal end of the distal body than the longitudinal portion of the protrusion.

Preferably, according to another embodiment, the sub-assembly of this invention can be used with a medicament delivery device comprising a medicament container containing a medicament.

Preferably, according to another embodiment, the medicament delivery device can be an injection device an inhalation device or a medical sprayer.

Preferably, according to another embodiment, when the medicament delivery device is an injector, the medicament delivery device can be an autoinjector.

Preferably, according to another embodiment, the medicament delivery device cassette unit is for accommodating the medicament container.

Preferably, according to another embodiment, the medicament delivery device with the sub-assembly as this invention can be operated by a method comprising the step of connecting the cassette unit to the drive unit.

Preferably, according to another embodiment, the method comprising the steps of connecting the cassette unit to the drive unit by rotating the cassette unit relative to the drive unit around the longitudinal axis.

A fourth aspect of the invention is provided with a method of assembling a medicament delivery device comprising: a cassette housing extending along a first longitudinal axis between a proximal end and a distal end, wherein the cassette housing defines an interior chamber; and an elongated plunger rod extending along a second longitudinal axis between a proximal end and a distal end, the method comprising the steps of: inserting a medicament container into the interior chamber of the cassette housing; covering the interior chamber from the distal end of the cassette housing with a distal cover, wherein the distal cover comprises a key slot perpendicular to the first longitudinal axis; attaching a key that is matched with the key slot to a proximal end of the plunger rod.

Preferably, according to another embodiment, the method further comprising the steps of: selecting a distal cover with a key slot in a certain geometry according to the medicament contained within the inserted medicament container before covering the interior chamber from the distal end of the cassette housing with the distal cover.

Preferably, according to another embodiment, the method further comprising the steps of: selecting a key with a certain geometry according to the medicament contained within the inserted medicament container before attaching the key to the proximal end of the plunger rod.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a sub-assembly of a medicament delivery device with a cassette housing and a distal body.

FIG. 2A schematically shows a perspective view of the cassette housing of the sub-assembly of FIG. 1.

FIG. 2B schematically shows a back view of a distal cover of the cassette housing of FIG. 2A.

FIGS. 3A-B schematically show perspective views of a distal body of the sub-assembly of FIG. 2.

FIG. 4 schematically shows a perspective view of a plunger rod with a key of the distal body of FIGS. 3a-3B.

FIG. 5 schematically show front views of key and/or key slot of FIG. 4 and/or FIG. 2B in differential geometry.

FIG. 6 schematically shows a perspective view of the cassette housing of FIG. 2A.

FIGS. 7A-B schematically show perspective views of the distal body of FIGS. 3A-B when the distal body is in an initial state.

FIG. 8 schematically shows a perspective view of the cassette housing of FIG. 2A aligned with the distal body of FIGS. 7A-B.

### DETAILED DESCRIPTION

The medicament delivery device cassette unit of the medicament delivery device is configured to contain: a medicament container containing a medicament and a medicament delivery member, e.g. a needle or a spray nozzle. Therefore, a cassette unit usually comprises a cassette housing and a medicament container.

Some of the cassette units may comprise a removable cap for covering the medicament delivery member (the removable cap is usually removably attached to the cassette housing), and some may comprise a pierceable sheath instead of the cap. The cassette unit may comprise a delivery member guard for surrounding the medicament delivery member. The cassette unit may be reusable or disposable. Some cassette units may contain the medicament container after a step of final assembly in a factory, and some may be shipped without containing a medicament container; instead, a caregiver or pharmacist may load a medicament container into the cassette unit before giving to a user.

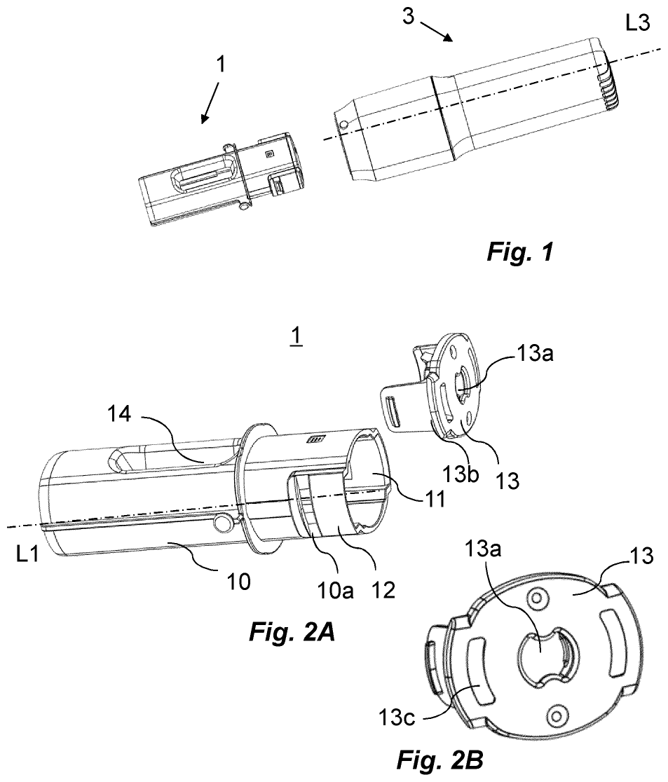

A first aspect of the invention provides a cassette housing 1 comprises a main body 10, a proximal engaging member 12 and a distal cover 13. Furthermore, FIGS. 1-7 illustrate the cassette housing 1 for a medicament delivery device cassette unit, the cassette housing comprising: the main body 10 extending along a first longitudinal axis L1 between a proximal end and a distal end. The main body 10 defines an interior chamber 11 for receiving a medicament container. The cassette housing comprises the distal cover 13 attached to the distal end of the main body 10, as shown in FIG. 2A. The distal cover 13 comprises covering wall extending across the distal end of the interior chamber 11 of the main body 10 in a direction transverse to the longitudinal axis L; and the covering wall 13 comprises a key slot 13a configured to receive a key 22 of a plunger rod 2 of the medicament delivery device, such that when the key 22 and the key slot 13a are matched, a key of the plunger rod 2 followed by a plunger rod body 21 of the plunger rod can enter into the cassette housing 1 through the key slot 13a. The key slot 13a is an aperture formed with a geometry corresponding a geometry of the key 22.

The cassette housing 1 can be used in a medicament delivery device cassette unit that only comprises (i.e. consists of) the cassette housing 1 and a medicament container; or further comprises one or more components dependent on the design of the cassette units. The medicament container can be a syringe or a cartridge; also, the medicament container can be made of glass or plastic, for example. The cassette housing 1 may further be used with a removable cap 15. The removable cap can be attached to the proximal end of the main body of the cassette housing, partially surrounding the main body of the cassette housing, as shown in FIG. 6 and FIG. 8, or fully enclosing the main body of the cassette housing.

The cassette housing may be formed with a generally tubular shaped main body, therefore an interior chamber 11, for receiving a medicament container of the medicament delivery device, can be defined, as shown in FIG. 2A. The exact shape of the cassette housing 1 can be formed in any other suitable shape that can contain the medicament container or with other functions, e.g. being compact or easy to hold. Further, the cassette housing 1 may comprise a proximal engaging member 12 attached to the distal portion of the main body 10 of the cassette housing 1.

When a medicament container is received in the interior chamber 11 of the cassette housing 1, the key slot 13a of the distal cover is aligned with a distal end of the medicament container; the plunger rod 2 for expelling the contained medicament can therefore pass through the key slot 13a and expel the contained medicament to the user. In a preferred example, the distal cover 13 is attached to the distal portion of the main body 10 of the cassette housing 1. In this example, the distal cover 13 comprises a connector 13b for fastening the distal cover 13 to a counter connector 10a of the main body 10 of the cassette housing 1 on the distal end of the main body 10 of the cassette housing 1. The engagement between the connector 13b of the distal cover 13 and the counter connector 10a of the cassette housing 1 can be a snap-fit connection, a bayonet connection or a thread connection, for example. The distal cover 13 optionally comprises one or more openings 13c; these openings can be provided if there is an element of the cassette unit that is designed to protrude through the distal end of the cassette housing 1. For example, the cassette unit may comprise a delivery member guard that will protrude from the proximal end of the cassette unit and interact with another part of the medicament delivery device. The one or more openings 13c are therefore formed in a shape dependent on the shape of the element of the cassette unit that is designed to protrude through the distal portion of the main body 10 of the cassette housing 1. Furthermore, the cassette housing 1 optionally comprises a window 14 on the main body 10, so that the user can observe the contained medicament from the window 14. Alternatively, the main body 10 of the cassette housing 1 can be transparent. In the example that the cassette housing 1 comprises the window 14 and the cassette housing is used with the removable cap 15, especially if the removable cap 15 partially or fully encloses the main body 10 of the cassette housing 1, the removable cap 15 may comprise a cap window 15a aligned with the window 14 of the cassette housing 1 when the removable cap 15 is attached to the main body 10 of the cassette housing 1, as shown in FIG. 6.

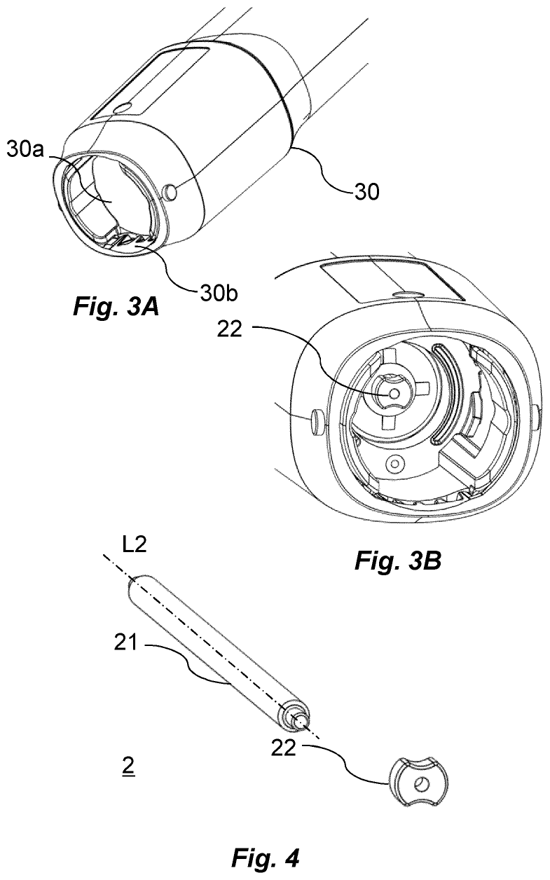

A second aspect of the invention provides a plunger rod 2 comprising a plunger rod body 21 and a key 22. Specifically, FIGS. 3A-7 illustrate the plunger rod 2 for a medicament delivery device, the plunger rod 2 comprising: the plunger rod body 21 extending along a second longitudinal axis L2 between a proximal end and a distal end; and the key 22. The key 22 is attached to the proximal end of the plunger rod body 21. The key 22 is configured to fit into a key slot 13a on a cassette housing, such that when the key 22 and the key slot 13a are matched, the key of the plunger rod 2 followed by the plunger rod body 21 can enter into the cassette housing 1 through the key slot 13a.

A dimension of a cross-section (observed along the second longitudinal axis L2) of the key 22 is greater than a dimension of a cross-section (observed along the second longitudinal axis L2) of the plunger rod body 21. Therefore, the plunger rod body 21 can only pass the key slot 13a of the distal cover 13 of the cassette housing 1 along the longitudinal axis L when the key passes the aperture 13a of the distal cover 13 of the cassette housing 1 along the longitudinal axis L first.

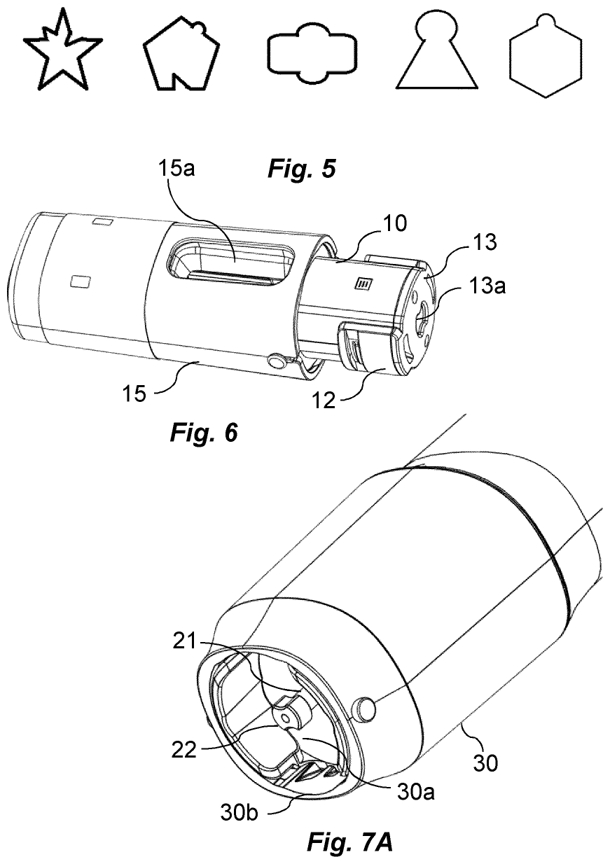

The key 22 can be integral with the plunger rod body 21 or be releasably attached to the plunger rod body 21. The key 22 is configured to match with the key slot 13a on the distal cover 13 of the cassette housing 1, meaning that when the key 22 passes the key slot 13a, the key 22 will be surrounded snugly by the key slot 13a. The key 22 and the key slot 13a can be formed by various geometries, for example, the front view of the key and the key slot can be formed as the geometries as shown in FIG. 5. Different geometries could be used for different medicaments to avoid accidental cross-compatibility.

A third aspect of the invention provides a sub-assembly of a medicament delivery device comprising the cassette housing 1 and the plunger rod 2 as defined above. The sub-assembly is configured to be used with a medicament delivery device comprising two parts, the cassette unit and a drive unit, that can be attached before carrying out a medicament delivery operation.

The drive unit of a medicament delivery device usually comprises an energy source and a plunger rod. The energy source is configured to connect to the plunger rod and provide a force applying on the plunger rod, so that the plunger rod can move into the cassette unit to expel the contained medicament. The energy source can be a motor, a spring, gas canister, or a force transfer component for being manually operated by a hand of the user.

The sub-assembly of the third aspect of the invention comprises the cassette housing 1, the plunger rod 2 and a distal body 3 configured to be attached to the cassette housing 1 and extending along a third longitudinal axis L3 between a proximal end and a distal end, as shown in FIG. 1. In a preferred example, the distal body 3 of the sub-assembly can be releasably attached to the cassette housing 1.

When the cassette housing 1 is attached to the distal body 3 or when the cassette housing 1 is axially aligned with the distal body 3 and thus ready for attaching to the distal body 3, the first longitudinal axis L1 of the cassette housing 1, the second longitudinal axis L2 of the plunger rod 2 and the third longitudinal axis L3 of the distal body 3 line up; in other words, the first, second and third longitudinal axis L1, L2, L3 should be the same longitudinal axis.

The distal body 3 of the sub-assembly is configured to be the housing of the drive unit of the medicament delivery device. Therefore, the plunger rod 2 and the energy source of the medicament delivery device are configured to be received within the distal body 3 of the sub-assembly. FIGS. 3A-3b and FIG. 4 illustrate that the distal body 3 comprises a main body 30. The plunger rod 2 as mentioned above is arranged within the main body 30 of the distal body 3 for expelling a medicament contained within the medicament container of the medicament delivery device. The plunger rod 2 can be connected to the energy source of the medicament delivery device. Therefore, when subjecting to a force from the energy source, the plunger rod 2 is axially movable, relative to the distal body 3, in the proximal direction of the distal body 3 from the proximal end of the distal body 3.

The distal body 3 may comprise a distal engaging member 30a, 30b attached to the proximal end of the distal body 3. The distal engaging member 30a, 30b is configured to interact with the proximal engaging member 12 of the cassette housing 1, so that the cassette housing 1 can be attached to the distal body 3 when the proximal engaging member 12 is fastened to the distal engaging member 30a, 30b. The connection formed between the proximal engaging member 12 and the distal engaging member 30a, 30b can be a bayonet connection, a thread connection or a snap-fit connection. For example, the proximal engaging member 12 can be fastened onto the distal engaging member 30a, 30b in the direction of the first longitudinal axis L1 through any of those above-mentioned exemplified connections. Further, the proximal engaging member can also be fastened to the distal engaging member in the direction of the first longitudinal axis L1 through the snap-fit connection. In one example, the proximal engaging member 12 can be a bayonet protrusion 12 and the distal engaging member 30a, 30b can be a bayonet slot (formed by one longitudinal portion 30a and one circumferential portion 30b). Therefore, the proximal engaging member 12 can be fastened to the distal engaging member 30a, 30b in the direction of the first longitudinal axis L1 through the bayonet connection. On the other hand, the cassette housing 1 can be attached to the distal body 3 through an independent connecting element. For example, the distal portion of the cassette housing 1 is aligned with the proximal end of the distal body 3; then a locking ring encloses both the distal portion of the cassette housing 1 and the proximal end of the distal body 3. The locking ring clamps tight on both cassette housing and the distal body, therefore the cassette housing is attached to the distal body. In this example, the sub-assembly doesn't need to comprise the proximal engaging member of the cassette housing and the distal engaging member of the distal body.

Because the key 22 on the plunger rod 2 in the distal body 3 will only fit into a corresponding key slot 13a on the distal cover 13 of the cassette housing 1, so that the key 22 on the plunger rod 2 in the distal body 3 and the key slot 13a on the distal cover 13 of the cassette housing 1 can therefore prevent a medicament delivery operation from being triggered when an unauthorized cassette unit of a medicament delivery device is accidentally attached to the drive unit of the medicament delivery device. For example, a caregiver can select a distal cover of the cassette housing that the distal cover comprises a certain geometry to define the key slot in accordance with a medicament container that has been loaded into the interior chamber of the cassette housing. Therefore, the medicament received within the cassette housing will only be expelled when the cassette housing is attached to a proper distal body.

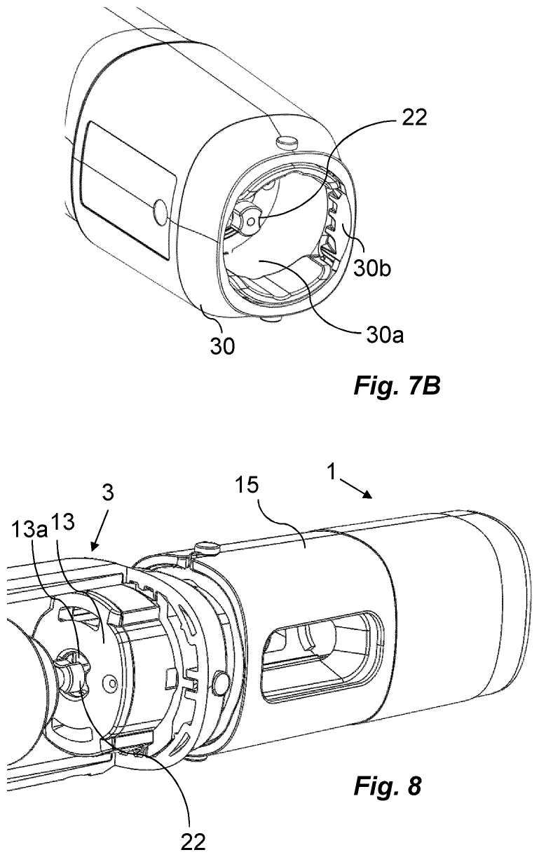

In another preferred example, the sub-assembly may provide an attaching prevention mechanism. When the sub-assembly comprises the proximal engaging member of the cassette housing and the distal engaging member of the distal body, the key and/or the key slot can be located at a certain position relative to the proximal engaging member and/or the distal engaging member. For example, the proximal engaging member 12 is positioned closer to the proximal end of the cassette housing 1 than a distal end of the distal cover 13, as shown in FIG. 6. The key 22 is protruding beyond at least a portion of the distal engaging member 30a, 30b in the proximal direction, as shown in FIG. 7. In this example, the proximal engaging member cannot reach to/fully engaged with the distal engaging member unless the key passes the key slot first. Therefore, if the user plans to attach a cassette unit of the medicament delivery device that comprises a key slot not matching with the key of the drive unit (meaning that the medicament received within that cassette unit should not be delivered by such drive unit), the user cannot attach that cassette unit to the drive unit.

For example, when the proximal engaging member 12 is a bayonet protrusion and the distal engaging member 30a, 30b is a bayonet slot, the key 22 of the plunger rod 2 is positioned beyond the longitudinal portion 30b of the bayonet slot 30a, 30b in the proximal direction, as shown in FIG. 7A. Alternatively, the key 22 of the plunger rod 2 is positioned extending through (or partially through) the circumferential portion 30a of the bayonet slot 30a, 30b, as shown in FIG. 7B. Therefore, the bayonet protrusion 12 cannot reach the circumferential portion 30a of the bayonet slot 30a, 30b so that the cassette housing cannot attach to the distal body unless the geometry of the key 22 of the plunger rod body 21 matches with the geometry of the key slot 13a on the distal cover 13 of the cassette housing 1.

In a further preferred example, the sub-assembly of the third aspect of the invention may further prevent the user from carrying out a medicament delivery operation when the drive unit is not ready for use, even the cassette unit is correct for being attached to the drive unit. For example, the drive unit may not yet be properly reloaded, e.g. a torsion spring needs tensioning when the torsion spring is the energy source; or the plunger rod needs pushing back to its initial position; or when the drive unit comprises a motor as the energy source, a battery level is low for driving a medicament delivery operation.

Some of the drive units of medicament delivery devices are reusable; that is, the drive units can expel the contained medicament multiple times and in multiple cassette units. Most of the reusable drive units of medicament delivery devices need to be reloaded after performing a single or certain times medicament delivery operation. The motion of reloading a drive unit may comprise the steps of moving the plunger rod to its initial position, tensioning the power spring (if the energy source is a spring) and/or reset a gear connected to a motor (if the energy source is a motor).

The function of preventing a medicament delivery operation when a drive unit that is not ready for use, e.g. the drive unit has not yet been properly reloaded, the drive unit reaches end of life, or the drive unit is self-diagnosed as broken, can be achieved by the key and the key slot arrangement. In this example, the plunger rod is movable (e.g. rotatable and/or linearly movable, will be explained in detail later) between a first position to a second position. In a preferred example, the plunger rod can be temporarily locked in the first position from being manually and directly moved by the user. For example, If the drive unit comprises a motor, the plunger rod typically can't be moved manually due to frictional resistance in the motor. Therefore, the user will not accidentally move the plunger rod from the first position to the second position if the drive unit is not ready, e.g. the battery for driving the motor is low, or the plunger rod has not yet been retracted back to the initial position by the motor. Furthermore, first position of the plunger rod can be chosen to position the plunger rod beyond the proximal end of the distal body, so that the user can easily observe the plunger rod in the first position, as an indication about the status of the drive unit, e.g. not ready for use.

For example, the plunger rod is rotatable around the longitudinal axis L relative to the distal body 3. The key 22 is rotationally fixed to the plunger rod body 21 so that the key 22 will rotate together with the plunger rod body 21. The plunger rod body 21 is rotatable around the third longitudinal axis L3 relative to the distal body 3 between a first position and a second position by a force from the energy source or the reloading motion. For example, the plunger rod 2 can be driven by the motor or driven through the reloading motion. When the plunger rod 2 is in the first position, the key 22 of the plunger rod 2 attached to the distal body 3 is misaligned with the key slot 13a of the distal cover 13 of the cassette housing 1. Therefore, the key 22 cannot pass the key slot 13a when the plunger rod 2 is in the first position so that the plunger rod 2 cannot pass the aperture 13a of the distal cover 13 of the cassette housing 1 either. A medicament operation is therefore prevented when the plunger rod 2 is in the first position. On the other hand, when the plunger rod 2 is in the second position, the key 22 is aligned with the key slot 13a, so that the plunger rod 2 can pass the aperture 13a of the distal cover 13 of the cassette housing 1 to expel a contained medicament received within the interior chamber 11 of the cassette housing 1. In other word, in this example, the key 22 is not matched to the key slot 13a when the plunger rod 2 is in the first position, and the key 22 is matched to the key slot 13a when the plunger rod 2 is in the second position.

Further, when the sub-assembly comprises the above-mentioned attaching prevention mechanism, the misalignment between the key 22 and the key slot 13a controlled by the rotatable plunger rod 2 can prevent the user from attaching a cassette unit of the medicament delivery device to the drive unit that is not ready for use. In this example, when the proximal engaging member is aligned with the distal engaging member in the direction of the third longitudinal axis L3 and the plunger rod is in the first position, the misalignment between the key 22 and the key slot 13a prevents the proximal engaging member from reaching to or fully engaging with the distal engaging member. The exact position of the first position of the plunger rod and/or second position of the plunger rod can be defined depending on the design of the medicament delivery device comprising the sub-assembly.

In another example, when the sub-assembly comprises the above-mentioned attaching prevention mechanism, instead of rotation, the plunger rod 2 is movable along the longitudinal axis in the distal direction of the distal body from a first position where to a second position. In this example, the key 22 is positioned relative to the key slot 13a when the plunger rod is either in the first position or the second position. In this example, the cassette housing 1 is configured to be fixed to the distal body 3 by a rotation, meaning that the connection between the proximal engaging member and the distal engaging member can be a bayonet connection or a thread connection. When the plunger rod 2 is in the first position, the proximal end of the plunger rod 2 is positioned closer to the proximal end of the distal body 3 than the distal engaging member 30a, 30b. When the plunger rod 2 is in the second position, the plunger rod 2 is positioned closer to the distal portion of the distal body 3 than the distal engaging member 30a, 30b. When the plunger rod 2 is in the first position and the proximal engaging member 12 is aligned with the distal engaging member 30a, 30b in the direction of the longitudinal axis, the key 22 of the plunger rod 2 in the distal body 3 is misaligned with the key slot 13a of the distal cover 13 of the cassette housing 1, as shown in FIG. 8. Therefore, the cassette housing 1 cannot attach to the distal body 3. When the plunger rod 2 is in the second position since the proximal engaging member 12 can reach and/or fully engage with the distal engaging member 30a, 30b, even the key 22 of the plunger rod 2 is misaligned with the key slot 13a, the user can still attach the cassette housing 1 to the distal body 3. The cassette housing 1 will attach to the distal body 3 by the rotation, the rotation of the cassette housing 1 also rotates the distal cover 13 and the key slot 13a of the cassette housing 1. Therefore, the key slot 13a can be aligned with the key 22 by the rotation for attaching the cassette housing 1 to the distal body 3. In the example that the proximal engaging member 12 of the cassette housing 1 is the bayonet protrusion and the distal engaging member 30a, 30b is a bayonet slot. When the plunger rod is in the second distal position, the proximal end of the plunger rod 2 is positioned closer to the distal end of the distal body 3 than the longitudinal portion 30b of the bayonet slot positioned relative to the distal body 3. Therefore, the bayonet protrusion 12 can be inserted into the bayonet slot through the longitudinal portion 30b of the bayonet slot; then the bayonet protrusion 12 can be rotated along the circumferential portion 30a of the bayonet slot to fix the cassette housing 1 to the distal body 3. When the bayonet protrusion 12 is aligned and/or in the longitudinal portion 30b of the bayonet slot, the key slot 13a is misaligned with the key 22. When the bayonet protrusion 12 rotates along the longitudinal portion 30b of the bayonet slot into a final fixed position, the key slot 13a is rotated to align with the key 22. Similarly, the exact position of the first position of the plunger rod and/or second position of the plunger rod can be defined depending on the design of the medicament delivery device comprising the sub-assembly. The plunger rod can be moved from the first position to the second position by the energy source of the medicament delivery device comprising the sub-assembly. Alternatively, the plunger rod can be moved from the first position to the second position together with the reloading motion.

It should be noted that, for achieving the above-mentioned attaching prevention mechanism, the key and the key slot are designed to allow the cassette housing to be inserted into the distal body and then rotated relative to the distal body, e.g. rotated 90 or 180 degrees, to allow a user (e.g. patient or care-giver) to attach the cassette housing into the distal body. The shaped geometry of the key and the key slot should be thus chosen based on the number of degrees that the cassette housing rotates relative to the distal body. For example, if 180 degrees is chosen, the geometry of the key and the geometry of the key slot should be symmetrical.

In a further example, the key 22 of the plunger rod 2 can be surrounded by the proximal end of the distal body 3 when the plunger rod 2 is in the first or the second position so that the user will not accidentally twist the key 22 of the plunger rod 2. Alternatively, the key 22 can be rotationally fixed to the plunger rod body 21, so that if the plunger rod is prevented from rotating when the plunger rod body 21 is in both the first and the second position, the key 22 will also be prevented from being twisted accidentally by the user.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are described in the clauses below.
1. A cassette housing (1) for a medicament delivery device cassette unit, the cassette housing (1) comprising:
   a main body (10) extending along a first longitudinal axis (L1) between a proximal end and a distal end; wherein the main body (10) defines an interior chamber (11) for receiving a medicament container;
   a distal cover (13) attached to the distal end of the main body (10);
   wherein the distal cover (13) comprises a covering wall extending across the distal end of the interior chamber (11) of the main body (10) in a direction transverse to the first longitudinal axis (L1); and
   wherein the covering wall comprises a key slot (13a) configured to receive a key (22) of a plunger rod (2) of the medicament delivery device, such that when the key (22) and the key slot (13a) are matched, the key (22) of the plunger rod (2) followed by a plunger rod body (21) of the plunger rod (2) can enter into the cassette housing (1) through the key slot (13a).
2. A plunger rod (2) for a medicament delivery device, the plunger rod (2) comprising:
   a plunger rod body (21) extending along a second longitudinal axis (L2) between a proximal end and a distal end;
   a key (22) attached to the proximal end of the plunger rod body (21);
   wherein the key (22) is configured to fit into a key slot (13a) on a cassette housing (1), such that when the key (22) and the key slot (13a) are matched, the key (22) followed by the plunger rod body (21) can enter into the cassette housing (1) through the key slot (13a).
3. A sub-assembly for a medicament delivery device, the sub-assembly comprising the cassette housing (1) according to clause 1 and the plunger rod (2) according to clause 2.
4. The sub-assembly according to clause 3, comprising a distal body (3) extending along a third longitudinal axis (L3) between a proximal end and a distal end; wherein the distal body (3) is configured be attached to the cassette housing (1); wherein the plunger rod (2) is arranged in the distal body (3) and is telescopic relative to the proximal end of the distal body (3).
5. The sub-assembly according to clause 4, wherein the cassette housing comprises a proximal engaging member attached at the distal end of the main body of the cassette housing; wherein the distal body comprises a distal engaging member attached to the proximal end of the distal body; and wherein the cassette housing is attached to the distal body when the proximal engaging member is fastened to the distal engaging member.
6. The sub-assembly according to clause 5, wherein the proximal engaging member is configured to be fastened to the distal engaging member in the direction of the third longitudinal axis (L3) through a bayonet connection.
7. The sub-assembly according to clause 6, wherein the proximal engaging member is a bayonet protrusion; and wherein the distal engaging member is a bayonet slot.
8. The sub-assembly according to any of clauses 5-7, wherein the proximal engaging member is positioned closer to the proximal end of the cassette housing than a distal end of the distal cover; and wherein the key of the plunger rod protrudes beyond at least a portion of the distal engaging member in the proximal direction of the distal body.
9. The sub-assembly according to clause 8 dependent on clause 7, wherein the key of the plunger rod protrudes beyond a longitudinal portion of the bayonet slot in the distal direction.
10. The sub-assembly according to any one of clauses 3-9, wherein the key of the plunger rod is rotationally fixed to the plunger rod body; and wherein the plunger rod body is rotatable around the second longitudinal axis (L2) from a first position to a second position.
11. The sub-assembly according to clause 10 dependent on any one of clauses 8-9, wherein the key of the plunger rod is misaligned with the key slot of the distal cover of the cassette housing in the circumferential direction relative to the first longitudinal axis (L1) when the plunger rod is in the first position and at least a portion of the proximal engaging member is aligned with the distal engaging member in the direction of the first longitudinal axis (L1).
12. The sub-assembly according to clause 10 dependent on clause 9, wherein the key of the plunger rod of the distal body is misaligned with the key slot of the distal cover of the cassette housing when the plunger rod is in the first position and the bayonet protrusion is aligned with the longitudinal portion of bayonet slot.
13. The sub-assembly according to any of clauses 3-12, wherein the distal body is configured to receive an energy source of the medicament delivery device, the energy source is configured to connect to the plunger rod for driving the plunger rod relative to the distal body.
14. The sub-assembly according to clause 13, wherein the energy source is a motor.
15. The sub-assembly according to the combination of any one of clauses 4-12 and one of clause 13-14, wherein the plunger rod is configured to move along the third longitudinal axis (L3) in the distal direction relative to the distal body from a first position to a second position when the energy source of the medicament delivery device received in the distal body has sufficient power to carry out a medicament delivery operation.
16. The sub-assembly according to clause 15 dependent on clause 9, wherein when the plunger rod is in the second distal position, the key of the plunger rod is positioned closer to the distal end of the distal body than the longitudinal portion of the bayonet slot.
17. A method of assembling a medicament delivery device comprising: a cassette housing extending along a first longitudinal axis (L1) between a proximal end and a distal end, wherein the cassette housing defines an interior chamber; and an elongated plunger rod extending along a second longitudinal axis (L2) between a proximal end and a distal end, the method comprising the steps of:
   inserting a medicament container into the interior chamber of the cassette housing;
   covering the interior chamber from the distal end of the cassette housing with a distal cover, wherein the distal cover comprises a key slot perpendicular to the first longitudinal axis (L1);
   attaching a key that is matched with the key slot to a proximal end of the plunger rod.
18. The method of assembling a medicament delivery device according to clause 17, the method further comprising the steps of:
   selecting a distal cover with a key slot in a certain geometry according to the medicament contained within the inserted medicament container before covering the interior chamber from the distal end of the cassette housing with the distal cover.
19. The method of assembling a medicament delivery device according to clause 17 or 18, the method further comprising the steps of:
   selecting a key with a certain geometry according to the medicament contained within the inserted medicament container before attaching the key to the proximal end of the plunger rod.

1. A cassette housing (1) for a medicament delivery device cassette unit, the cassette housing (1) comprising:
a main body (10) extending along a first longitudinal axis (L1) between a proximal end and a distal end; wherein the main body (10) defines an interior chamber (11) for receiving a medicament container;
a distal cover (13) attached to the distal end of the main body (10);
wherein the distal cover (13) comprises a covering wall extending across the distal end of the interior chamber (11) of the main body (10) in a direction transverse to the first longitudinal axis (L1); and
wherein the covering wall comprises a key slot (13a) configured to receive a key (22) of a plunger rod (2) of the medicament delivery device, such that when the key (22) and the key slot (13a) are matched, the key (22) of the plunger rod (2) followed by a plunger rod body (21) of the plunger rod (2) can enter into the cassette housing (1) through the key slot (13a).

2. A plunger rod (2) for a medicament delivery device, the plunger rod (2) comprising:
a plunger rod body (21) extending along a second longitudinal axis (L2) between a proximal end and a distal end;
a key (22) attached to the proximal end of the plunger rod body (21);
wherein the key (22) is configured to fit into a key slot (13a) on a cassette housing (1), such that when the key (22) and the key slot (13a) are matched, the key (22) followed by the plunger rod body (21) can enter into the cassette housing (1) through the key slot (13a).

3. A sub-assembly for a medicament delivery device, the sub-assembly comprising:
a cassette housing (1) comprising:
a main body (10) extending along a first longitudinal axis (L1) between a proximal end and a distal end; wherein the main body (10) defines an interior chamber (11) for receiving a medicament container;
a plunger rod body (21) extending along a second longitudinal axis (L2) between a proximal end and a distal end;
a key (22) attached to the proximal end of the plunger rod body (21);
a distal cover (13) attached to the distal end of the main body (10);
wherein the distal cover (13) comprises a covering wall extending across the distal end of the interior chamber (11) of the main body (10) in a direction transverse to the first longitudinal axis (L1); and
wherein the covering wall comprises a key slot (13a) configured to receive the key (22) of the plunger rod (2), such that when the key (22) and the key slot (13a) are matched, the key (22) of the plunger rod (2) followed by plunger rod body (21) of the plunger rod (2) can enter into the cassette housing (1) through the key slot (13a).

4. The sub-assembly according to claim 3, comprising a distal body (3) extending along a third longitudinal axis (L3) between a proximal end and a distal end; wherein the distal body (3) is configured be attached to the cassette housing (1); wherein the plunger rod (2) is arranged in the distal body (3) and is telescopic relative to the proximal end of the distal body (3).

5. The sub-assembly according to claim 4, wherein the cassette housing comprises a proximal engaging member attached at the distal end of the main body of the cassette housing; wherein the distal body comprises a distal engaging member attached to the proximal end of the distal body; and wherein the cassette housing is attached to the distal body when the proximal engaging member is fastened to the distal engaging member.

6. The sub-assembly according to claim 5, wherein the proximal engaging member is configured to be fastened to the distal engaging member in the direction of the third longitudinal axis (L3) through a bayonet connection.

7. The sub-assembly according to claim 6, wherein the proximal engaging member is a bayonet protrusion; and wherein the distal engaging member is a bayonet slot.

8. The sub-assembly according to claim 5 wherein the proximal engaging member is positioned closer to the proximal end of the cassette housing than a distal end of the distal cover; and wherein the key of the plunger rod protrudes beyond at least a portion of the distal engaging member in the proximal direction of the distal body.

9. The sub-assembly according to claim 3, wherein the key of the plunger rod is rotationally fixed to the plunger rod body; and wherein the plunger rod body is rotatable around the second longitudinal axis (L2) from a first position to a second position.

10. The sub-assembly according to claim 9, wherein the key of the plunger rod is misaligned with the key slot of the distal cover of the cassette housing in the circumferential direction relative to the first longitudinal axis (L1) when the plunger rod is in the first position and at least a portion of the proximal engaging member is aligned with the distal engaging member in the direction of the first longitudinal axis (L1).

11. The sub-assembly according to claim 3, wherein the distal body is configured to receive an energy source of the medicament delivery device, the energy source is configured to connect to the plunger rod for driving the plunger rod relative to the distal body.

12. The sub-assembly according to claim 4, wherein the plunger rod is configured to move along the third longitudinal axis (L3) in the distal direction relative to the distal body from a first position to a second position when the energy source of the medicament delivery device received in the distal body has sufficient power to carry out a medicament delivery operation.

13. A method of assembling a medicament delivery device comprising: the cassette housing according to claim 1, and an elongated plunger rod extending along a second longitudinal axis (L2) between a proximal end and a distal end, the method comprising the steps of:
inserting a medicament container into the interior chamber of the cassette housing;
covering the interior chamber from the distal end of the cassette housing with a distal cover, wherein the distal cover comprises a key slot perpendicular to the first longitudinal axis (L1);
attaching a key that is matched with the key slot to a proximal end of the plunger rod.

14. The method of assembling a medicament delivery device according to claim 13, the method further comprising the steps of:
selecting a distal cover with a key slot in a certain geometry according to the medicament contained within the inserted medicament container before covering the interior chamber from the distal end of the cassette housing with the distal cover.

15. The method of assembling a medicament delivery device according to claim 13, the method further comprising the steps of:
selecting a key with a certain geometry according to the medicament contained within the inserted medicament container before attaching the key to the proximal end of the plunger rod.
